(19) United States
(12) Patent Application Publication
(10) Pub. No.: US 2024/0290460 A1
(43) Pub. Date: Aug. 29, 2024

(54) PERSONAL INSIGHTS PLATFORM THAT EDUCATES AND EMPOWERS USERS THROUGH PREDICTIVE ANALYTICS OF THEIR HABITUAL BEHAVIOR

(71) Applicants: Kaladhar Peddinti, Fairfax, VA (US); Srimaye Peddinti, Fairfax, VA (US)

(72) Inventors: Kaladhar Peddinti, Fairfax, VA (US); Srimaye Peddinti, Fairfax, VA (US)

(21) Appl. No.: 18/176,039

(22) Filed: Feb. 28, 2023

(51) Int. Cl. G16H 20/60; G06N 5/022

(52) U.S. Cl. CPC G16H 20/60; G06N 5/022

(57) ABSTRACT

A system for habitual behavior data collection and the artificial intelligence analysis thereof for data interpretation and associated insightful output, the system embodied in a platform having a user interface module for collecting intrinsic descriptive analytical data comprising at least and a software interface module for receiving extrinsic descriptive analytical data from a plurality of computer devices, wherein the extrinsic descriptive analytical data at least partially derived from a habitual behavior output having associated purchase inputs indicative of a purchase thereof. The platform inputs the intrinsic and extrinsic descriptive analytical data into a trained artificial intelligence module, the artificial intelligence module having been trained using a machine learning algorithm having as input habitual behavior training data, so as to generate insightful output.

### BACKGROUND OF THE INVENTION

The present invention relates to intelligence analytics engines, and, more particularly, a personal insights platform that educates and empowers users through predictive analytics of their habitual behavior.

Two routines of behavior (or habits) that are repeated regularly and tend to occur subconsciously are food and spending habits. Both types of behavior require a lot of personal intelligence (when done mindfully) that is not tapped effectively because of the person's fixed way of thinking, willing, or feeling acquired through previous repetition of the mental experience of consuming calories and spending dollars, whereby such behavior often goes unnoticed in the person exhibiting it, because a person does not ‘need’ to engage in self-analysis when undertaking such “routine” tasks.

Question: how much did you spend on electricity, cell phone, gas, and services in the last year?

Take the above to a deeper level: how much protein, carbohydrates, fat, sugar, oil did you buy? What were their food sources? What did it cost?

Collecting and analyzing this information could lead to a healthier, more effective diet plan and lifestyle. The resulting descriptive analytical data may be shared with one's medical care provider, dietitian, nutritionist, or other qualified entity for rendering helpful diagnostics or diagnostic analytical data. This diagnostic analytical data then can, in turn, be processed to determine potential outcomes of present actions derived from the diagnostic analytical data, thereby generating predictive analytical data, which enables behavioral change (as opposed to mindless habitual behavior that is suboptimal, detrimental, and possibly self-destructive).

Connecting this self-awareness (the aggregation of descriptive analytical data, diagnostic analytical data, and predictive analytical data) can result in personal analytics forming the basis for what the inventor calls the ‘Quantitative Life’, or personal intelligence analytics mentioned above. Such personal intelligence analytics can be applied to a user's future decision tree choices, thereby impacting future actions big and small, from choice of recipes to social media interactions to setting a carbon footprint. As the amount of ‘Quantitative Lives’ increases, global statistics and a changing world will ensue.

There are online platforms that provide “news” regarding information about the world. There are also “social network platforms” covering most of the social aspects of life. But what about personal intelligence platforms? At this moment there is some personal information analysis in the shape of Mint™, American Express™, Bank of America™, and other credit cards, where they categorize/summarize card member spending and present that member with some analytics, but not to the level of insights that can educate and empower people to negotiate for themselves because current information analysis does not involve predictive analytics.

Google™ enabled users to “search” for information, Twitter™ enabled users to be “chatty”, Facebook™ enabled users to be “social”, Wikipedia™ enabled users to be “intelligent”, Amazon™ enabled users to be “online shoppers”. But there is no singular online platform for “quantifying one's life”.

As can be seen, there is a need for a personal insights platform that educates and empowers users through predictive analytics of their habitual behavior, wherein the personal insights platform helps people to harness information from their habits (spending, utilization, etc.) and provides insights to educate and empower them regarding future actions, thereby quantifying the user's life.

### SUMMARY OF THE INVENTION

In one aspect of the present invention, a system for generating predictive analytics of a habitual behavior, the system includes a processor, and a memory comprising computing device-executable instructions that, when executed by the processor, cause the processor to implement: a communications interface for accessing an insight platform over a network; a user interface for displaying and interacting with the insight platform; a user interface module for collecting intrinsic descriptive analytical data regarding the habitual behavior on the insight platform, a software interface module for receiving extrinsic descriptive analytical data regarding the habitual behavior on the insight platform, wherein the software is integrated with application programming interfaces (API); and an artificial intelligence module for generating an insightful output based in the intrinsic and extrinsic intrinsic descriptive analytical data, wherein the insightful output is a predictive outcome for the habitual behavior.

In another aspect of the present invention, the system for generating predictive analytics of a habitual behavior further includes wherein the habitual behavioral is spending on a food product, wherein the collected intrinsic descriptive analytical data is a scanned receipt for the food product, wherein the received extrinsic descriptive analytical data is a calorie amount associated with the food product, and wherein the calorie amount is collected through a public API, wherein the insightful output is embodied in an exportable report providing a nutritional analysis and a spending analysis, wherein the spending analysis is based on the scanned receipt, and wherein the nutritional analysis is based on the calorie amount.

In another aspect of the present invention, the system for generating predictive analytics of a habitual behavior further includes wherein the artificial intelligence module has been trained using a machine learning algorithm having input habitual behavior training data, wherein the artificial intelligence module is configured to render a diagnostic analytical dataset for each descriptive analytical data, and wherein the diagnostic analytical dataset is in turn used to generate the insightful output, wherein the diagnostic analytical dataset is derived through a correlation algorithm, by way of the processor, wherein the correlation algorithm utilizes key value pairs to determine a weight for each descriptive analytical data, wherein the weight is based on a rating provided by a third-party entity, received by way of the processor.

These and other features, aspects and advantages of the present invention will become better understood with reference to the following drawings, description, and claims.

### BRIEF DESCRIPTION OF THE DRAWINGS

FIG. 1 is a block diagram of an exemplary embodiment of the present invention.

FIG. 2 is a block diagram of an exemplary embodiment of the present invention.

FIG. 3 is schematic view of a computing device of an embodiment of the present invention.

FIG. 4 is a schematic view of a network of computing devices of an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a system for habitual behavioral data collection and the artificial intelligence analysis thereof for data interpretation and associated insightful output. The present invention is embodied in a platform having a (i) user interface module for collecting intrinsic descriptive analytical data comprising; and (ii) a software interface module for receiving extrinsic descriptive analytical data from a plurality of computer devices, wherein the extrinsic descriptive analytical data is at least partially derived from a habitual behavioral output having associated purchase inputs indicative of a purchase. The platform inputs the intrinsic and extrinsic descriptive analytical data into a trained artificial intelligence module, the artificial intelligence module having been trained using a machine learning algorithm having as input habitual behavior training data, so as to generate insightful output.

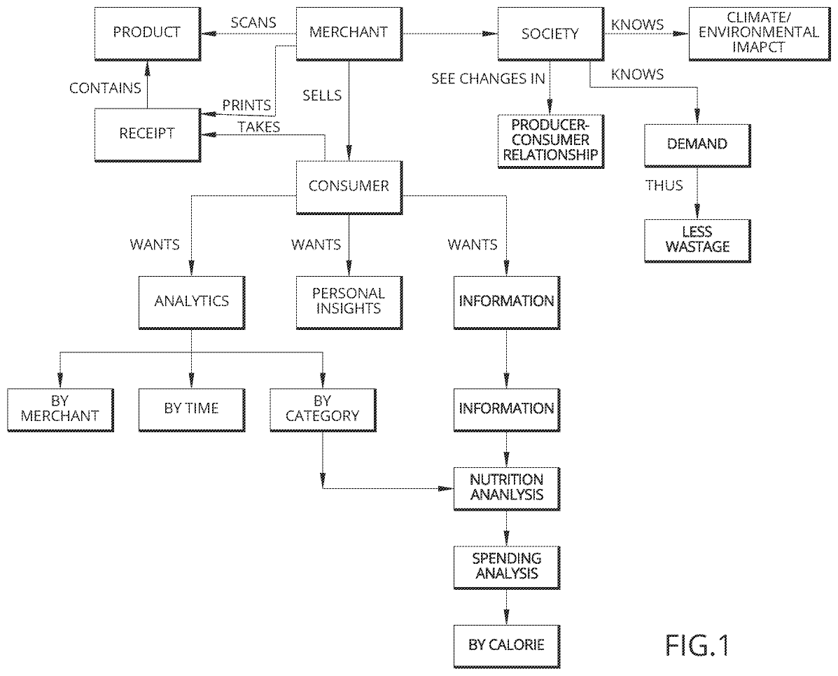

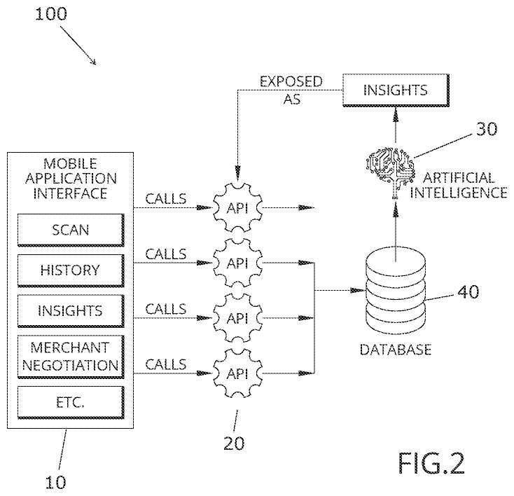

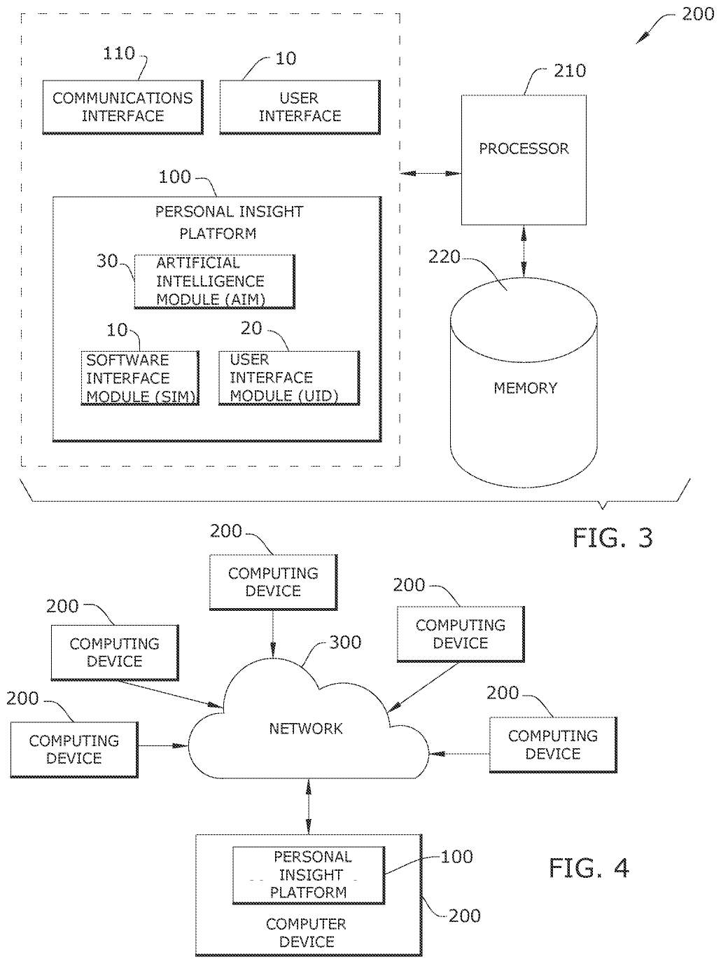

Referring now to FIGS. 1 through 4, the present invention may include a system for personal insight comprising a processor 210, and a memory comprising computing device-executable instructions that, when executed by the processor 210, cause the processor to implement: a communications interface for accessing a person insight platform 100 over a network 300. The personal insight platform 100 comprises a front-end user interface module (UIM) 10 coupled to software interface (SIM) 20. The SIM 20 may include one or more application programming interfaces (APIs) whereby one or more intrinsic computer programs of the UIM can communicate with one or more extrinsic computer programs. UIM 10 enables users of the platform to enter habitual information and perform descriptive analytics on said habitual information, resulting in descriptive analytic data. The SIM 20 enables extraction of extrinsic descriptive analytic data.

The personal insight platform 100 further comprises an Artificial Intelligence module (AIM) 30 that provides insightful output in the form of predictive analytical data composed from said descriptive analytic data. Diagnostic analytic data creation may happen within the AIM 30 as a precursor to the predictive analytical data. It being understood that the predictive analytical data persists on all modules 10, 20, (and 30) as well as being retrievably stored in a database 40.

UIM 10 and SIM 20 are used to gather and determine intrinsic and extrinsic descriptive analytical data. Data pipelines prepare descriptive analytical data for the AIM 30 to extract personal insights in the form of predictive analytical data. The personal insight output is adapted to educate the user and enable the user to negotiate transactions regarding consumable and durable goods.

In one embodiment, the UIM 10 and SIM 20 may extract the various data from various data points, including but not limited to receipts and other points of sale the user. The personal insight platform 100 may convert that data into JavaScript Object Notation (JSON) with key value pairs to determine each expense. Key value pairs are Name Value pairs used in data collection and storage. An example of “key value pairs” would be “Category Name, Number of Items”; “Item Name, Item Value” etc. The SIM 20 may be configured to render the descriptive analytic data points into fundamental components of the domain by way of the key value pair, creating a domain ontology. The components of the domain ontology may further comprise a set of learning domain states and elements identifying a level of association for each descriptive analytic data point. The SIM 20 may employ a mathematical algorithm that helps to identify how strongly data are related to one another through use of the principal components of the dataset. The algorithm finds patterns in high dimensional data where a simple graphical representation is not feasible. Through SIM 20, a third-party entity (e.g., a nutritionist accessing another computing device 200, as illustrated in FIG. 4) could be given similarity metrics that would rate some behavior in terms of their similarity with other behavior, wherein the rating is associated with the data. The algorithm could eigenvectors to transform a set of third-party observations of possibly correlated variables into linearly uncorrelated variables. These correlated variables may be the basis for diagnostic analytic datasets.

The SIM 20 may integrate with point of sale or scanned receipts to capture the spending receipt information. The SIM 20 may include API integration with merchandise point of sale. For instance, if it is a food item, a public API may be accessed via the SIM 20 to obtain calories etc.

When there is sufficient descriptive analytical data, as determined by AIM 30, the AIM 30 may run routines to extract diagnostic and predictive analytical data to render personal insight output based on, in part, the extracted descriptive analytical data.

A method of using the present invention may include downloading a software application or using the web platform embodying the personal insight platform 100, then the user may, by way of example and by no means limiting, uploads data from behavior output sources, e.g., receipts of spending from points of sale, via the UIM 10. The personal insight platform 100 integrates the information and data from the behavior output sources where possible to extract the descriptive analytical data directly. The AIM 30 harvests descriptive analytical data and therefrom generates diagnostic and predictive analytical data.

The present invention provides a system for habitual behavior collection and analysis and artificial intelligence analysis thereof for predictive analytic data interpretation and associated personal insight output which transcends prior art systems.

As will be described in further detail below, the system collects habitual behavior data of users from a plurality of behavior output sources associated with habitual behavior, and the trained AIM 30 provides various artificial intelligence insights useful to such users which may further modify habitual behavior.

As will be described in further detail below, the trained AIM 30 has a data interpretation controller configured for intelligently interpreting descriptive analytical data collected through the output sources.

Furthermore, the trained AIM 30 can, in embodiments, generate intelligent insightful notifications which may be helpful for the user. Such insightful notifications may be further derived from the machine learning algorithm.

According to one aspect, there is provided a system for habitual behavior data collection and the artificial intelligence analysis thereof for data interpretation and associated insightful output, the system comprising: a user interface module for collecting intrinsic descriptive analytical data comprising at least a user ID data; and a software interface module for: receiving extrinsic descriptive analytical data from a plurality of computer devices, the extrinsic descriptive analytical data at least partially derived from a habitual behavior output having associated purchase inputs indicative of a purchase, inputting the intrinsic and extrinsic descriptive analytical data into a trained artificial intelligence module, the artificial intelligence module having been trained using a machine learning algorithm having as input habitual behavior training data.

The AIM 30 trained artificial intelligence analytics engine may comprise a data interpretation controller which processes descriptive analytical data fed into the trained AIM 30 trained. Having input such data, the trained AIM 30 configured to generate intelligent artificial intelligence insightful output, which may then be conveyed back to relevant electronic devices 200 of the network 300. Such insightful outputs are any intelligent data and/or notifications which may be useful to user when engaging in future habitual behavior.

In embodiments, the trained AIM 30 may take the form of an artificial neural network and therefore the trained data may represent the optimized weightings for each node of the neural network.

In sum, the personal insight platform 100 provides a framework to harvest any input data and gain insights using AIM 30 so that the user is a true king, where producers are chasing for the consumer buying power. The input data can be sourced from external market and/or vendor data API, files, etc., as well as databases (app related or external)

Database 40 stores the analytical data in the form of data packages or files accessible to the server 210. In one preferred embodiment, database 40 is a relational database that maintains the data packages files in a search pool. In another preferred embodiment, the database 40 is a directory server, such as a Lightweight Directory Access Protocol (‘LDAP’) server, that maintains the data packages files in the search pool. In other implementations, the database 40 and search pool are a configured area in the non-volatile memory 220 of the server 210 that maintains the data packages files information.

Communication interface 110 manages communications between the access (computing) devices 200 and the server 210. Possibly, the communication interface 110 may include a web server configured to send and receive information in the form of web pages to any of the browsers or applications in response to a request, wherein the web server communicates with each web browser or application and software interface module (SIM) 20 using one or more communication protocols, such as HTTP (Hyper Text Markup Language). In one preferred embodiment, for example, the web server is configured to include the Apache HTTP Server from the Apache Software Foundation. In another preferred embodiment, the web server includes Internet Information Services (IIS) from Microsoft Corporation. In yet another preferred embodiment, the web server includes the Sun Java System Web Server from Sun Microsystems.

The software application may be based on mobile technologies such as Flutter, React JS, Node JS, Mobile App Servers etc. Can we extend the technology section to add these technologies and generalize to include all Web, App ad Mobile technologies.

In certain embodiments, the network 300 may refer to any interconnecting system capable of transmitting audio, video, signals, data, messages, or any combination of the preceding. The network may include all or a portion of a public switched telephone network (PSTN), a public or private data network, a local area network (LAN), a metropolitan area network (MAN), a wide area network (WAN), a local, regional, or global communication or computer network such as the Internet, a wireline or wireless network, an enterprise intranet, or any other suitable communication link, including combinations thereof.

The server and the computer of the present invention may each include computing systems. This disclosure contemplates any suitable number of computing systems. This disclosure contemplates the computing system taking any suitable physical form. As example and not by way of limitation, the computing system may be a virtual machine (VM), an embedded computing system, a system-on-chip (SOC), a single-board computing system (SBC) (e.g., a computer-on-module (COM) or system-on-module (SOM)), a desktop computing system, a laptop or notebook computing system, a smart phone, an interactive kiosk, a mainframe, a mesh of computing systems, a server, an application server, or a combination of two or more of these. Where appropriate, the computing systems may include one or more computing systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

In some embodiments, the computing systems may execute any suitable operating system such as IBM's zSeries/Operating System (z/OS), MS-DOS, PC-DOS, Mac-OS, Windows, Unix, OpenVMS, an operating system based on Linux, or any other appropriate operating system, including future operating systems as well as mobile operating systems such as Android and IOS etc. In some embodiments, the computing systems may be a web server running web server applications such as Apache, Microsoft's Internet Information Server™, and the like.

In particular embodiments, the computing systems include a processor, a memory, a user interface and a communication interface. In particular embodiments, the processor includes hardware for executing instructions, such as those making up a computer program. The memory includes main memory for storing instructions such as computer program(s) for the processor to execute, or data for processor to operate on. The memory may include mass storage for data and instructions such as the computer program. As an example and not by way of limitation, the memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to computing system, where appropriate. In particular embodiments, the memory is non-volatile, solid-state memory.

The user interface may include hardware, software, or both providing one or more interfaces for communication between a person and the computer systems. As an example, and not by way of limitation, a user interface device may include a keyboard, keypad, microphone, monitor, mouse, printer, scanner, speaker, still camera, stylus, tablet, touchscreen, trackball, video camera, another suitable user interface or a combination of two or more of these. A user interface may include one or more sensors. This disclosure contemplates any suitable user interface.

The communication interface includes hardware, software, or both providing one or more interfaces for communication (e.g., packet-based communication) between the computing systems over the network. As an example, and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface. As an example, and not by way of limitation, the computing systems may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing systems may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing systems may include any suitable communication interface for any of these networks, where appropriate.

It should be understood, of course, that the foregoing relates to exemplary embodiments of the invention and that modifications may be made without departing from the spirit and scope of the invention as set forth in the following claims.

What is claimed is:

1. A system for generating predictive analytics of a habitual behavior, the system comprising:
a processor, and
a memory comprising computing device-executable instructions that, when executed by the processor, cause the processor to implement:
a communications interface for accessing an insight platform over a network;
a user interface for displaying and interacting with the insight platform;
a user interface module for collecting intrinsic descriptive analytical data regarding the habitual behavior on the insight platform,
a software interface module for receiving extrinsic descriptive analytical data regarding the habitual behavior on the insight platform, wherein the software is integrated with application programming interfaces (API); and
an artificial intelligence module for generating an insightful output based in the intrinsic and extrinsic intrinsic descriptive analytical data, wherein the insightful output is a predictive outcome for the habitual behavior.

2. The system of claim 1, wherein the habitual behavioral is spending on a food product, wherein the collected intrinsic descriptive analytical data is a scanned receipt for the food product, wherein the received extrinsic descriptive analytical data is a calorie amount associated with the food product, and wherein the calorie amount is collected through a public API.

3. The system of claim 2, wherein the insightful output is embodied in an exportable report providing a nutritional analysis and a spending analysis.

4. The system of claim 3, wherein the spending analysis is based on the scanned receipt, and wherein the nutritional analysis is based on the calorie amount.

5. The system of claim 1, wherein the artificial intelligence module has been trained using a machine learning algorithm having input habitual behavior training data.

6. The system of claim 5, wherein the artificial intelligence module is configured to render a diagnostic analytical dataset for each descriptive analytical data, and wherein the diagnostic analytical dataset is in turn used to generate the insightful output.

7. The system of claim 6, wherein the diagnostic analytical dataset is derived through a correlation algorithm, by way of the processor, wherein the correlation algorithm utilizes key value pairs to determine a weight for each descriptive analytical data.

8. The system of claim 7, wherein the weight is based on a rating provided by a third-party entity, received by way of the processor.
